(19) United States
(12) Patent Application Publication
(10) Pub. No.: US 2011/0166598 A1
(43) Pub. Date: Jul. 7, 2011

(54) DEVICES AND METHODS FOR TONGUE STABILIZATION

(75) Inventors: Donald A. Gonazles, San Antonio, TX (US); Anthony Natale, New Preston, CT (US); Fred B. Dinger, III, San Antonio, TX (US)

(73) Assignee: ENTRIGUE SURGICAL, INC., San Antonio, TX (US)

(21) Appl. No.: 12/958,957

(22) Filed: Dec. 2, 2010

Related U.S. Application Data
(60) Provisional application No. 61/266,035, filed on Dec. 2, 2009.

(51) Int. Cl. A61B 17/04

(52) U.S. Cl. 606/228; 606/232

(57) ABSTRACT

Tongue-stabilization devices and methods, such as, for example, for treatment and/or reduction of symptoms of obstructive sleep apnea (OSA).

### CROSS-REFERENCE TO RELATED APPLICATIONS

This application claims priority to U.S. Provisional Patent Application Ser. No. 61/266,035 (filed Dec. 2, 2009), incorporated by reference herein.

### BACKGROUND

1. Field of the Invention

The present invention relates generally to devices and methods for stabilizing the tongue of a mammal, such as a human, and, more particularly, but not by way of limitation, to devices and methods for stabilizing the tongue to prevent and/or alleviate symptoms of obstructive sleep apnea (OSA).

2. Description of Related Art

Obstructive sleep apnea (OSA) is disease that affects up to 20% of the adult population. OSA generally occurs during sleep when soft tissue obstructs the airway and creates cessation of or impedes breathing. Obstruction can occur at one or more levels including the retropalatal and retrolingual areas. Surgical correction of such obstructions remains a challenge, specifically for the retrolingual area. Removal or ablation of tongue tissue has been utilized with poor results due to complications, such as severe bleeding, abscess formation, and/or the inability to move the tongue anterior enough to relieve the obstruction.

A number of devices and methods have been developed and/or are in use in the art for the treatment, and/or alleviation of symptoms of obstructive sleep apnea (OSA). The following references involve examples of methods or devices related to OSA, and may facilitate understanding of background information and possible application-specific information for this and related fields of endeavor: (1) U.S. patent application Ser. No. 10/597,590, filed Feb. 28, 2005, and published as Pub. No. US 2009/0014012; (2) U.S. patent application Ser. No. 12/011,782, filed Jan. 29, 2008, and published as Pub. No. US 2008/0188947; (3) U.S. patent application Ser. No. 11/762,642, filed Jun. 13, 2007, and published as Pub. No. US 2008/0023012; (4) U.S. patent application Ser. No. 11/835,931, filed Aug. 8, 2007, and published as Pub. No. US 2008/006769; (5) U.S. patent application Ser. No. 11/820,168, filed Jun. 18, 2007, and published as Pub. No. US 2008/0066864; (6) U.S. patent application Ser. No. 11/762,752, filed Jun. 13, 2007, and published as Pub. No. US 20008/0058584; (7) U.S. patent application Ser. No. 11/835,966, filed Aug. 8, 2007, and published as Pub. No. US 2008/0083461; (8) U.S. patent application Ser. No. 11/598,220, filed Nov. 9, 2006, and published as Pub. No. US 2007/0144539; (9) U.S. patent application Ser. No. 11/107,160, filed Apr. 15, 2005, and published as Pub. No. US 2006/0235264; (10) U.S. patent application Ser. No. 11/334,216, filed Jan. 18, 2006, and published as Pub. No. US 2007/0163603 (11) U.S. patent application Ser. No. 10/746,707, filed Dec. 23, 2003, and published as Pub. No. US 2005/0133026; and (12) U.S. Pat. No. 6,408,851, filed Oct. 1, 1991. The foregoing references numbered (1) through (12) are hereby incorporated by reference in their entireties.

### SUMMARY

The present disclosure includes various embodiments of devices and methods for tongue stabilization, such as, for example, for treatment and/or alleviation of symptoms of OSA.

Some embodiments of the present tongue stabilization devices, comprise: an anchor having an anterior penetration point, a posterior end, and a plurality of barbs each having a point extending away from the penetration point; a suture connected to the anchor; and a counter-tension member configured to be slidably disposed on the suture. In some embodiments, the counter-tension member is configured to freely slide relative to the suture in a first direction, and to resist sliding relative to the suture in a second direction. In some embodiments, at least one of the anchor, suture, and counter-tension member comprises a bio-absorbable material. In some embodiments, each of the anchor, suture, and counter-tension member each comprise a bio-absorbable material.

Unlike existing tongue stabilization devices, embodiments of the present tongue stabilization devices do not require attachment to the mandible. As explained in more detail below, during use of exemplary embodiments the removal or incision in the tongue can cause either reduction of tongue tissue or the reorientation of the incision by creating tension on the suture and pulling the posterior aspect of the tongue anteriorly. The creation of a wound can allow the tongue to heal in the new orientation so tension is only needed during the healing process and not permanently. The temporary nature of the use of exemplary embodiments can allow for its components (e.g. the anchor, suture, and counter-tension member) to be constructed from bioabsorbable material.

Certain existing devices that are configured to be anchored to the mandible can require constant and permanent tension to effectively reposition or stabilize the tongue. Such configurations are susceptible to breakage due to the constant and permanent tension placed on the components. Exemplary embodiments of the present disclosure do not require anchoring to the mandible and are not as susceptible to breakage because the tongue can be stabilized or repositioned without permanent tension. In certain exemplary embodiments, multiple devices can be placed in the tongue, spreading the tension among the different implants and sculpting the base of tongue, thereby further reducing the likelihood of breakage.

In exemplary embodiments, the device is entering through a clean wound, reducing the risk of infection and abscess formation (in contrast to existing methods of using on radio frequency ablation on the base of tongue through the surface). With exemplary embodiments, the closure of the wound can also help create hemostasis which can reduce the possible complication of a hematoma formation. Furthermore, since the surface of the tongue is not penetrated, pain may be greatly reduced.

Some embodiments of the present methods of stabilizing a tongue of a patient, comprise: providing a tongue-stabilization device (e.g., comprising: an anchor configured such that if the anchor is inserted into soft tissue of a patient the anchor will resist removal from the soft tissue; a suture having a first end connected to the anchor, and a second end extending from the anchor; and a counter-tension member configured to be slidably disposed on the suture); creating an incision through the skin in the submental (under the mandible) area; inserting the tongue-stabilization device through the incision such that the anchor is disposed near a posterior portion of the tongue and the suture extends through the anterior portion of the tongue and through the incision; positioning the counter-tension member along the suture outside the tongue adjacent a lower surface of the tongue; and tensioning the suture between the anchor and the counter-tension member.

In some embodiments, inserting comprises: disposing at least a portion of the tongue-stabilization device in a needle; inserting the needle through the incision such that the anchor is disposed near a posterior portion of the tongue and the needle extends through the anterior portion of the tongue and through the incision; and removing the needle from the tongue.

Certain embodiments may further comprise: creating a void in the tongue proximal to the anchor, where the void is oriented generally parallel to an axis extending from the posterior portion of the tongue to the anterior portion of the tongue prior to tensioning the suture; and the void is oriented generally perpendicular to an axis extending from the posterior portion of the tongue to the anterior portion of the tongue after tensioning the suture.

In some embodiments, inserting comprises: disposing at least a portion of the tongue-stabilization device in an insertion device, wherein the insertion device comprises an ablation mechanism; inserting the insertion device through the incision such that the anchor is disposed near a posterior portion of the tongue and the insertion device extends through the anterior portion of the tongue and through the incision; ablating tissue with the ablation mechanism; and

removing the insertion device from the tongue. In certain embodiments, the ablative tissue is anterior to the anchor such that when the counter-tension member is placed in the suture the wound is closed by moving the posterior tongue anteriorly. The ablation of tissue can be accomplished by an energy source such as ultrasound, radio frequency, electricity, etc. The ablation may create the reduction of tongue tissue, or an anterior to posterior (AP) incision. When tension is placed on the suture the wound may close in a posterior to anterior orientation or the AP incision may change orientation to lateral. The closure or reorientation of the wound can result in anterior movement of the base of tongue. In certain embodiments, the movement can become permanent once the tissue heals in the new orientation.

In some embodiments, the step of tensioning the suture is performed before the step of positioning the counter tension member. In some embodiments, the step of positioning the counter-tension member comprises sliding the counter-tension member along the suture. In some embodiments, the counter-tension member is configured such that if the counter-tension member is disposed on the suture the counter-tension member will freely slide relative to the suture in a first direction, and resist sliding relative to the suture in a second direction. In some embodiments, the anchor has an anterior penetration point, a posterior end, a longitudinal axis extending from the, and a plurality of barbs each having a point extending away from the penetration point. In some embodiments, the suture comprises a plurality of barbs extending away from the anchor. In some embodiments, the anchor, suture, and counter-tension member each comprise a bio-absorbable material.

Some embodiments of the present tongue-stabilization devices, comprise: a body having an elongated portion with a coupling end and an enlarged end, the body configured to be inserted into a hole through the tongue of a human patient, and the enlarged end configured to resist being pulled through the hole; and a retention member having a coupling portion and a tooth portion, the coupling portion configured to be coupled to the coupling end of the body, and the tooth portion configured to fit between a set of two laterally-adjacent teeth of the patient. In certain embodiments, the retention member may be configured to attach or couple to the teeth or on the teeth. In specific embodiments, the retention member may be a custom-fitted, removable dental appliance that fits over the teeth, a permanent appliance that is cemented or otherwise affixed directly to the teeth or any device that rests between the teeth or slips around one tooth or multiple teeth.

In some embodiments, the coupling end of the body comprises an opening, the coupling portion of the retention member comprises a ring configured to be coupled to the opening. In some embodiments, the ring is configured to be removably coupled to the opening. In some embodiments, the retention member comprises a narrow portion configured to fit between two laterally-adjacent teeth, and an enlarged portion configured to not fit between the two laterally-adjacent teeth. As noted above, in certain embodiments, the retention member may be configured to attach or couple to the teeth or on the teeth.

In some embodiments, the retention member comprises two tooth portions each configured to fit between a different set of two laterally-adjacent teeth of the patient. In some embodiments, each tooth portion comprises a narrow portion configured to fit between two laterally-adjacent teeth, and an enlarged portion configured to not fit between the two laterally-adjacent teeth. In some embodiments, the retention member is configured is configured such that if worn by a patient at least a portion of the retention member extends linearly across the lower jaw of the patient substantially perpendicular to the elongated portion of the body. In some embodiments, the two tooth portions of the retention member are unitary, and the coupling portion is the midpoint between the enlarged portions of the two tooth portions.

In some embodiments, the body and the retention member are unitary. In some embodiments, the coupling portion of the body comprises an enlarged portion configured to resist movement of the tongue of a patient relative to the body. In some embodiments, the coupling end of the body has a maximum transverse dimension that is substantially the same as or less than the maximum transverse dimension as the elongated portion of the body. Some embodiments further comprise: a coupling member configured to be coupled to the coupling end of the body and the coupling portion of the retention member. In some embodiments, the coupling member comprises chain.

In some embodiments, the body is configured to extend through at least two portions of the tongue of a patient. In some embodiments, the elongated portion and coupling end of the body are each flexible. In some embodiments, the elongated portion and coupling end of the body are unitary. In some embodiments, the elongated portion and coupling end of the body comprise chain. In some embodiments, the retention member comprises chain.

Some embodiments of the present methods of stabilizing a tongue of a patient, comprise: providing a tongue-stabilization device (e.g., comprising: a body having an elongated portion with a coupling end and an enlarged end, the body configured to be inserted into a hole through the tongue of a human patient, and the enlarged end configured to resist being pulled through the hole; and a retention member having a coupling portion and a tooth portion, the coupling portion configured to be coupled to the coupling end of the body, and the tooth portion configured to couple to one or more teeth or to fit between a set of two laterally-adjacent teeth of the patient); positioning the body of the tongue-stabilization device through a hole in the tongue of a patient; coupling the coupling portion of the retention member to the coupling end of the body of the tongue-stabilization device; and positioning each of the one or more tooth portions of the retention member between a set of two laterally-adjacent teeth of the patient such that the tongue-stabilization device resists posterior motion of the tongue. Other embodiments may comprise attaching the retention member directly to the teeth or on the teeth.

Some embodiments of the present tongue-shaping devices comprise: a bar configured to extend transversely across the tongue of a patient through two holes in the tongue.

Any embodiment of any of the present methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 depicts a side cross-sectional view of a human patient having one embodiment of the present tongue-stabilization devices implanted the tongue of the patient.

FIG. 2A depicts an anchor and suture of the embodiment of FIG. 1 with an insertion needle.

FIG. 2B depicts an anchor and suture of the embodiment of FIG. 1 with an insertion needle.

FIG. 3 depicts an anchor of the embodiment of FIG. 1.

FIG. 4 depicts a counter-tension device of the embodiment of FIG. 1.

FIG. 5 depicts perspective view of a barbed suture suitable for use with or in some embodiments of the present tongue-stabilization devices and methods.

FIGS. 6A-6E depicts certain aspects of some embodiments of the present tongue-stabilization methods and methods of implanting the present tongue-stabilization devices.

FIGS. 7 and 8 depict side views of portions of two embodiments of tongue-stabilization devices in the tongue of a human patient.

FIGS. 9-23 depict various views of embodiments of the present tongue-stabilization devices.

FIG. 24 depicts a front view of one of the present tongue-shaping devices.

### DESCRIPTION OF ILLUSTRATIVE EMBODIMENTS

The term “coupled” is defined as connected, although not necessarily directly, and not necessarily mechanically; two items that are “coupled” may be integral with each other. The terms “a” and “an” are defined as one or more unless this disclosure explicitly requires otherwise. The terms “substantially,” “approximately,” and “about” are defined as largely but not necessarily wholly what is specified, as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps. For example, in a method that comprises providing a tongue-stabilization device, the method includes the specified steps but is not limited to having only those steps. For example, such a method could also include inserting the device through an incision into the tongue of a patient.

Further, a device or structure that is configured in a certain way is configured in at least that way, but it can also be configured in other ways than those specifically described.

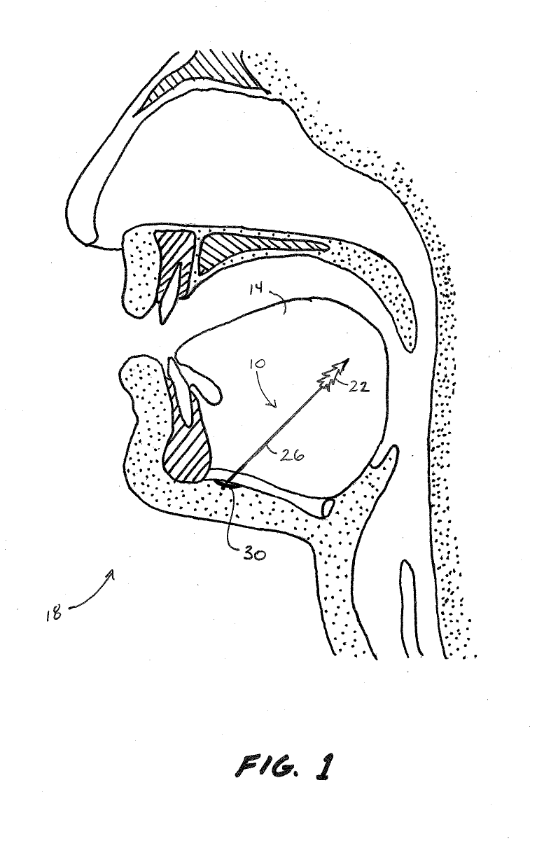

Referring now to the drawings, and more particularly to FIG. 1, shown therein and designated by the reference numeral 10 is one of the present tongue-stabilization devices. More specifically, device 10 is shown implanted in a tongue 14 of a human patient 18. In the embodiment shown, device 10 comprises an anchor 22, a suture 26, and a counter-tension member 30. Anchor 22 is configured such that if the anchor is inserted into soft tissue (e.g., of tongue 14) of a patient the anchor will resist removal from the soft tissue (e.g., of tongue 14). Device 10 can be (and is shown) configured to take advantage of the tongue being naturally anchored anteriorly to the mandible. For example, device 10 can be implanted to compress the tongue in an anterior-posterior direction such that the natural tethering between the tongue and mandible will pull the tongue anteriorly (forward).

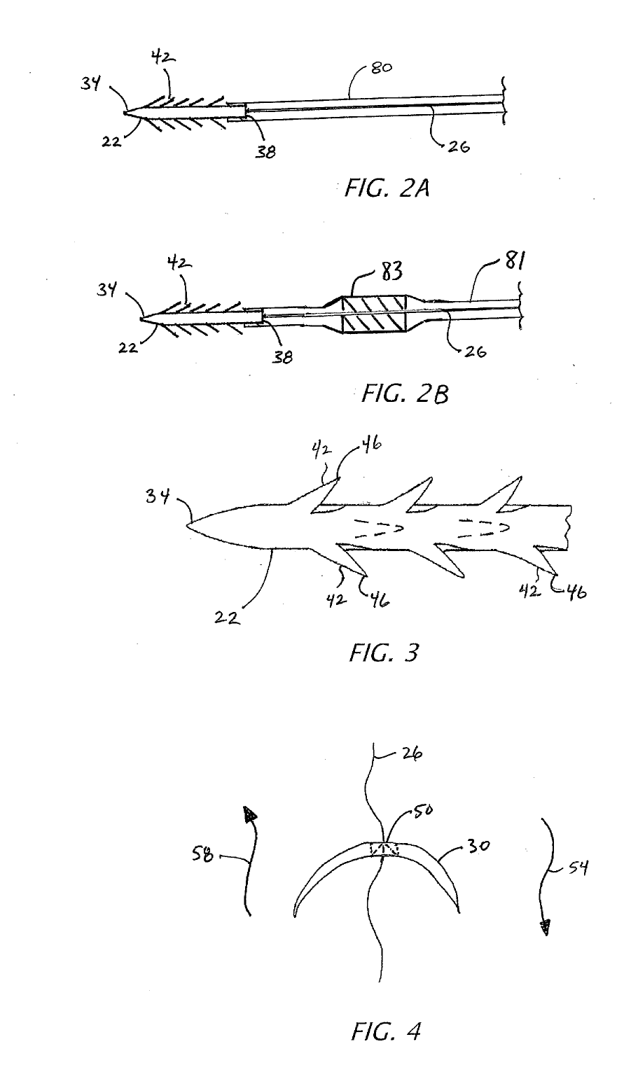

Referring now to FIGS. 2A, 2B, 3, and 4, anchor 22, suture 26, and counter-tension member 30 are shown in more detail. Anchor 22 has an anterior penetration point 34, a posterior end 38, and a plurality of barbs 42 each having a point 46 extending away from the penetration point 34. In the embodiment shown in FIG. 2A, suture 26 extends through an insertion needle 80 and is coupled (e.g., connected) to anchor 22, such as, for example, at posterior end 38 of anchor 22 (e.g., by way of a knot, adhesive, and/or the like).

The embodiment shown in FIG. 2B is similar to that shown in FIG. 2A, with the exception that suture 26 extends through an insertion device 81 that comprises a tissue ablation mechanism 83. Tissue ablation mechanism 83 may comprise any one of a number of mechanisms configured to ablate tissue, including for example, an transmitter of radio or ultrasonic frequency electromagnetic waves. In other non-limiting examples of tissue ablation mechanism 82 may comprise a laser transmitter, or mechanical devices (e.g., a microdebrider).

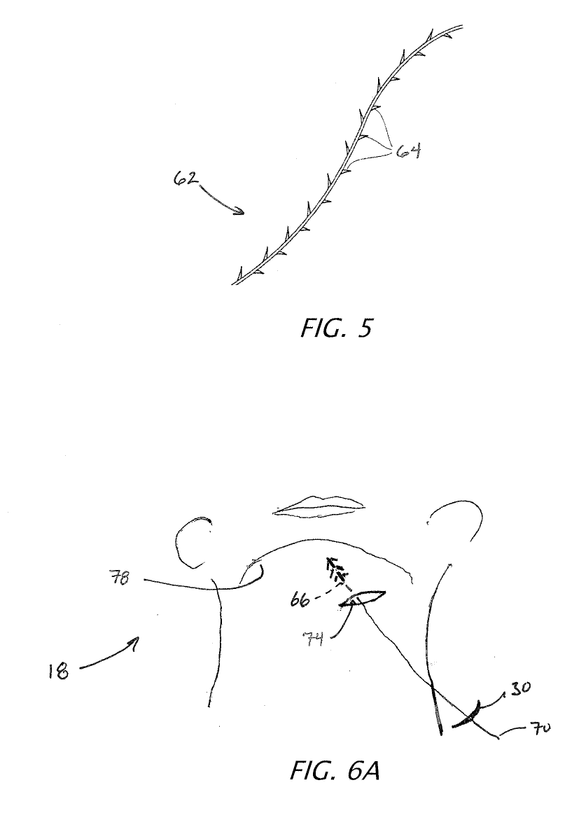

Counter-tension member 30 is configured to be slidably disposed on suture 26, such as, for example, by way of an opening 50 configured such that suture 26 fits through opening 50 of counter-tension member 30. In the embodiment shown in FIG. 5, counter-tension member 30 is configured to freely slide relative to the suture in a first direction 54, and to resist sliding relative to the suture in a second direction 58. In this way, as is described in more detail below, counter-tension member 30 can be slid along suture 26 into an installed position (e.g., as in FIG. 1) and counter-tension member 30 will resist sliding out of the installed position (e.g., will resist releasing any tension in suture 26 between counter-tension member 30 and anchor 22). The counter-tension member can be configured (with or by any suitable structure, assembly, or the like) to freely slide relative to the suture in a first direction 54, and to resist sliding relative to the suture in a second direction 58. For example, counter-tension member 30 can include a tapered cone structure having aperture 50 through it that is configured to increase the size of aperture 50 when a force is applied to counter-tension member 30 in first direction 54, and to decrease the size of aperture 50 and thereby pinch suture 22 (to resist sliding) when a force is applied to counter-tension member 30 in second direction 58. In other embodiments, suture 26 can be wrapped or tied around counter-tension member 30 once counter-tension member 30 is in a desired position relative to suture 26 and/or tongue 14 (e.g., to maintain a tension in suture 26). In some embodiments, counter-tension member 30 is provided with a protrusion, notch, spool, or other structure (not shown) to facilitate wrapping and/or tying of the suture to the counter-tension member.

In some embodiments, one or more (e.g., one, all, etc.) of anchor 22, suture 26, and/or counter-tension member 30 each comprises a bio-absorbable material. Anchor 22, suture 26, and/or counter-tension member 30 can comprise any suitable biocompatible material. In certain exemplary embodiments, anchor 22, suture 26, and/or counter-tension member 30 comprise an absorbable copolymer comprising approximately 60 to 80 percent polyactide and approximately 20 to 40 percent polyglycolide. More specifically, anchor 22, suture 26, and/or counter-tension member 30 may comprise an absorbable copolymer comprising approximately 65 to 75 percent polyactide and approximately 25 to 35 percent polyglycolide. In a specific exemplary embodiment, anchor 22, suture 26, and/or counter-tension member 30 comprises an absorbable copolymer comprising approximately 70 percent polyactide and approximately 30 percent polyglycolide. In other embodiments, anchor 22, suture 26, and/or counter-tension member 30 may be non-absorbable (e.g., may comprise a non-absorbable material).

FIG. 5 depicts a barbed suture 62 that can be used with various embodiments of the present tongue stabilization devices. In some embodiments, the barbed suture is integral to anchor 22. Barbed suture 62 comprises a plurality of barbs 64. In some embodiments, barbed suture is coupled to anchor 22 in place of suture 26 such that that barbs 64 extend away from anchor 22. Barbed suture 62 can comprise any of the materials described above for anchor 22, suture 26, and/or counter-tension member 30.

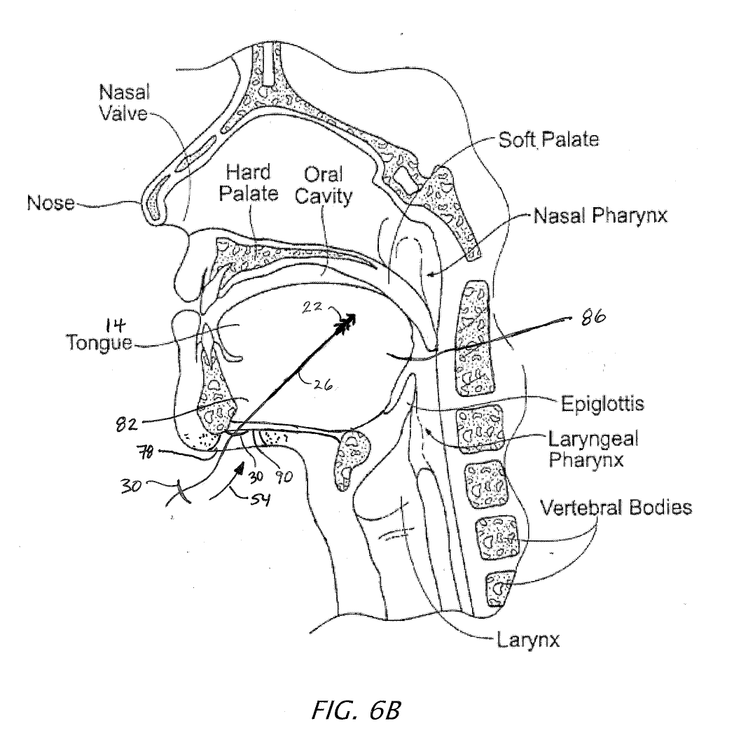

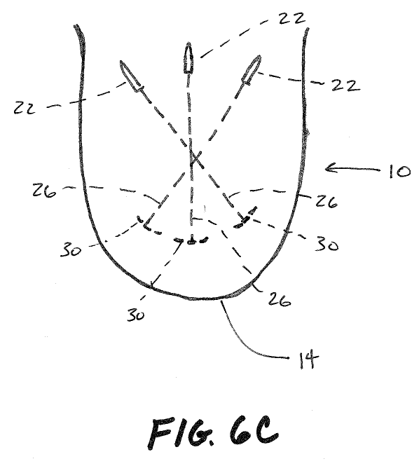

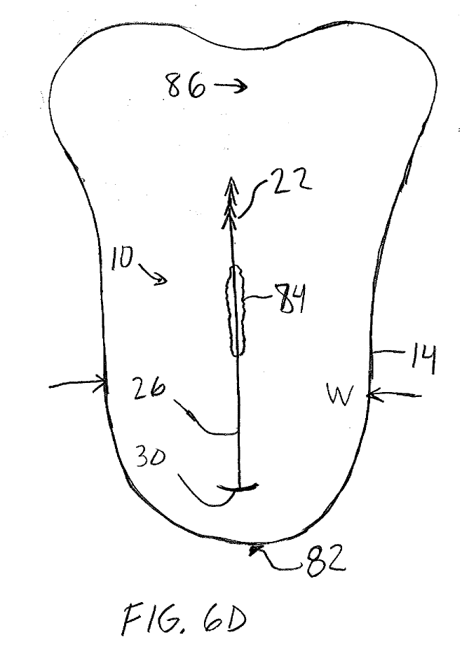

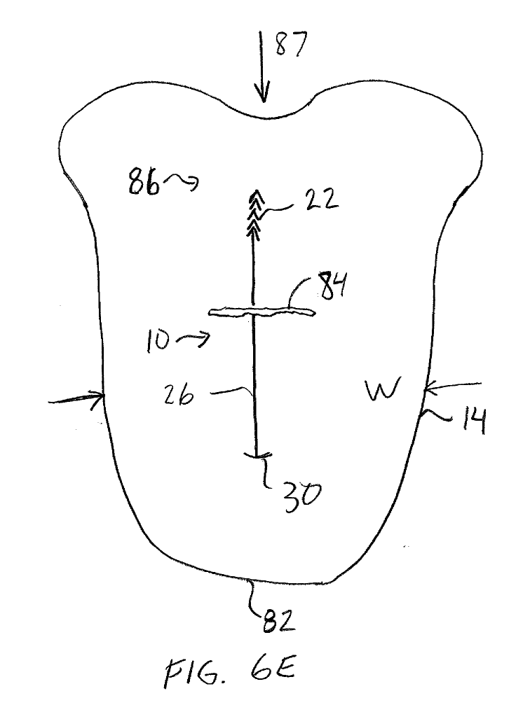

Referring now to FIGS. 6A-6E, one method of stabilizing a tongue of a patient is illustrated. In the embodiment illustrated, the method comprises: providing a tongue-stabilization device that comprises: an anchor 22 configured such that if the anchor is inserted into soft tissue (e.g., of tongue 14) of a patient the anchor will resist removal from the soft tissue; a suture 26 having a first end 66 connected to anchor 22, and a second end 70 extending from anchor 22; and a counter-tension member 30 configured to be slidably disposed on suture 26. The method can further comprise creating an incision 74 through the skin on the lower side of the patient's jaw 78, and inserting anchor 22 of the device 10 through incision 74 (e.g., with insertion needle 80 shown in FIG. 2A or insertion device 81 shown in FIG. 2B) and into an anterior portion 82 of tongue 14 toward a posterior portion 86 of tongue 14. In some embodiments, anchor 14 is inserted into tongue 14 such that anchor 14 is disposed near posterior portion 86 of tongue 14 and suture 26 extends through anterior portion 82 of tongue 14 and through incision 78. The method can further comprise positioning counter-tension member 30 along suture 26 outside tongue 14 adjacent a lower surface 90 of tongue 14 (e.g., by sliding counter-tension member 30 along suture 26 in direction 54 toward lower surface 90 of tongue 14). The method can further comprise tensioning suture 26 between anchor 22 and counter-tension member 30. Stated another way, once counter-tension member 30 is positioned adjacent lower surface 90 of tongue 14, suture 26 can be pulled through counter-tension member 30 to pull posterior portion 86 of the tongue forward to, for example, open the airway, shape the rear of the tongue, stabilize the tongue, or the like.

Stated another way, device 10 can be placed minimally invasively by making a small incision 74 under the mandible, inserting a placement device (e.g., needle with suture internal, etc.) such that suture 26 extends out of the tongue anteriorly (e.g., through an anterior portion 82 of the tongue); and placing a counter-tension member 30 on suture 26 that can only move along suture 26 in an anterior to posterior direction. By placing tension on suture 26 while pushing counter-tension member 30 in a posterior direction (toward posterior portion 86 of the tongue) against the tissue of the tongue, suture 26 will compress the tongue between anchor 22 and counter-tension member 30 and pull at least a portion of the tongue in an anterior direction. Multiple barbs 42 on anchor 22 (and/or barbed suture 62) can spread the forces of the tension in the suture, and multiple anchors 22 can spread the forces of the tension on the suture among multiple regions of the tongue so that each barb has a relatively minimal amount of tension.

In some embodiments of the present methods, inserting anchor 22 comprises: disposing at least a portion of the anchor (e.g., posterior end 38) in a needle; inserting the needle through incision 74 such that the anchor is disposed near posterior portion 86 of the tongue and the needle extends through anterior portion 82 of the tongue and through incision 74; and removing the needle from the tongue. For example, insertion needle 80 comprises a distal end 92 configured to be temporarily coupled to anchor 22 for insertion of anchor 22 into soft tissue (e.g., tongue 14) of the patient. Needle 80 also comprises a proximal or base end (not shown) that can be coupled to any suitable handle, actuator, or the like, such as, for example, that can be configured to decouple or eject anchor 22 from distal end 92 of needle 80. In the embodiment shown, distal end 92 stops short of penetration point 34 of anchor 22 such that penetration point 34 facilitates or enables penetration during insertion of anchor 22, and/or such that upon insertion of anchor 22 into tongue 14, barbs 42 are permitted to engage the soft tissue of the tongue such that needle 80 can be removed from tongue 14 while anchor 22 will resist removal from the soft tissue (e.g., such that anchor 22 will remain inserted or embedded in the soft tissue of the tongue). In other embodiments, distal end 92 can be configured to extend past penetration point 34 of anchor 22 (and/or distal end 92 can be angled and/or sharpened as in a traditional hypodermic needle) such that distal end 92 facilitates or enables insertion of needle 80 and anchor during insertion of anchor 22. In some embodiments, needle 80 can be configured to cover and/or at least partially compress barbs 42 while anchor 22 is coupled to and/or disposed within needle 80 (e.g., during insertion of a anchor 22 into tongue 14. In the embodiment shown, needle 80 is configured such that if anchor 22 is coupled to or disposed within needle 80 then suture 26 extends through at least a portion (and/or the entire length of) needle 80. In other embodiments, needle 80 can comprise a notch or opening configured to permit suture 26 to pass through the notch or opening rather than extending the length of the needle.

Referring now to FIGS. 6D and 6E, the orientation of a void 84 in tongue 14 is altered as tension is placed on suture 26 via anchor 22 and counter-tension member 30. In certain embodiments, void 84 can be formed in tongue 14 at least partially circumferentially around device 10 and proximal to anchor 22. In specific embodiments void 84 can be formed via an incision in tongue 14 or by ablating tissue in tongue 14 (e.g., via issue ablation mechanism 83). In specific embodiments, void 84 may be formed via tissue ablation and suction. Upon initial placement of device 10 into tongue 14, void 84, is oriented generally parallel to an axis extending from posterior portion 86 of tongue 14 to anterior portion 82 of tongue 14. This configuration is shown in FIG. 6D.

After tension is placed on suture (e.g., by pushing counter-tension member 30 in a posterior direction toward posterior portion 86 the orientation of void 84 is altered so that it is generally perpendicular to an axis extending from posterior portion 86 of tongue 14 to anterior portion 82 of tongue 14. This configuration is shown in FIG. 6E. In addition to altering the orientation of void 84, the tension on suture 26 also increases the width of tongue 14 (e.g., the dimension illustrated by dimension “W” in FIGS. 6D and 6E). Furthermore, the tension on suture 26 may also decrease the thickness of tongue 14 (e.g. the dimension of tongue 14 measured in the same direction of suture 26 as shown in FIG. 6B) and move posterior portion 86 of tongue 14 in the direction indicated by arrow 87 in FIG. 6E.

In certain embodiments, a suture passer may be utilized to create a continuous (e.g. running) suture by passing needle 80 between the arms of the suture passer. In still other embodiments, suture 26 may be welded together to maintain a low profile and reduce the patient's sensation of suture 26.

In some embodiments of the present methods, insertion device 81 shown in FIG. 2B, rather than needle 80, may be utilized to insert anchor 22 and suture 26. Insertion device 81 can be inserted into tongue 14 in a manner similar to that of previously described embodiments (e.g., through a submental incision). Ablation mechanism 83 can then be used to ablate tissue of tongue 14 (e.g., via a radio or ultrasound frequency electromagnetic wave transmitter, laser transmitter, microdebrider, shaver, or any other suitable mechanism).

Once the tissue is ablated, insertion device 81 can be withdrawn leaving suture 26 in place. Suture 26 can be pulled, while anchor 22 (and counter-tension member 30) keep tension on suture 26. This can collapse the ablated area, pulling tongue 14 tissue anteriorly, e.g. so that the thickness of tongue 14 in the ablated area is decreased while the width (measured across the mandible) is increased. In certain embodiments, insertion device 81 may also be configured to provide suction to remove the ablated tissue. In specific embodiments, ablation mechanism 83 is sufficiently spaced from anchor 22 so that, after installation, anchor 22 will be located in tissue that has not been ablated. The above-described technique does not require an anchor to mandible, allowing multiple sutures to be placed in the tongue. In specific embodiments, anchor 22, suture 26, and counter-tension member 30 may be absorbable since the tissue will heal in the new configuration and there is no need to rely on continued tension to cause the desired effect.

The above-described technique can also provide several advantages over other known ablative techniques, which generally require penetrating through the surface and seeding the area with bacteria. Such techniques generally require the surgeon to wait for the tissue to remodel to create the desired effect after the ablation is completed.

In some embodiments of the present methods, barbed suture 62 can be used in place of suture 26, or in place of a portion of suture 26. For example, device 10 can comprise a length of barbed suture 62 adjacent anchor 22, and a length of suture 26 coupled to (e.g., unitary with) the length of barbed suture 62, such that the length of barbed suture 62 is configured to assist anchor 22 in maintaining the position of anchor 22 relative to tongue 14, and the length of suture 26 is configured to permit retention member 30 to slide on the length of suture 26. In some embodiments, the step of tensioning the suture is performed before the step of positioning the counter tension member. That is, in some embodiments, after anchor 22 is inserted into tongue 14, suture 26 is tensioned before counter-tension member 30 is positioned adjacent lower surface 90 of the tongue. In this way, counter-tension member 30 can be positioned once the suture is tensioned.

In some embodiments of the present methods, the anchor can comprise anchor 22 of FIGS. 1-3. In other embodiments, the anchor can comprise any suitable anchor that is configured to resist removal once inserted into soft tissue. In some embodiments, the present methods can comprise a plurality of tongue-stabilization devices (e.g., 10) to shape and/or stabilize the tongue of a patient in a suitable and/or desired way. For example, FIG. 6C depicts a tongue 14 with three separate devices 10 inserted into the tongue.

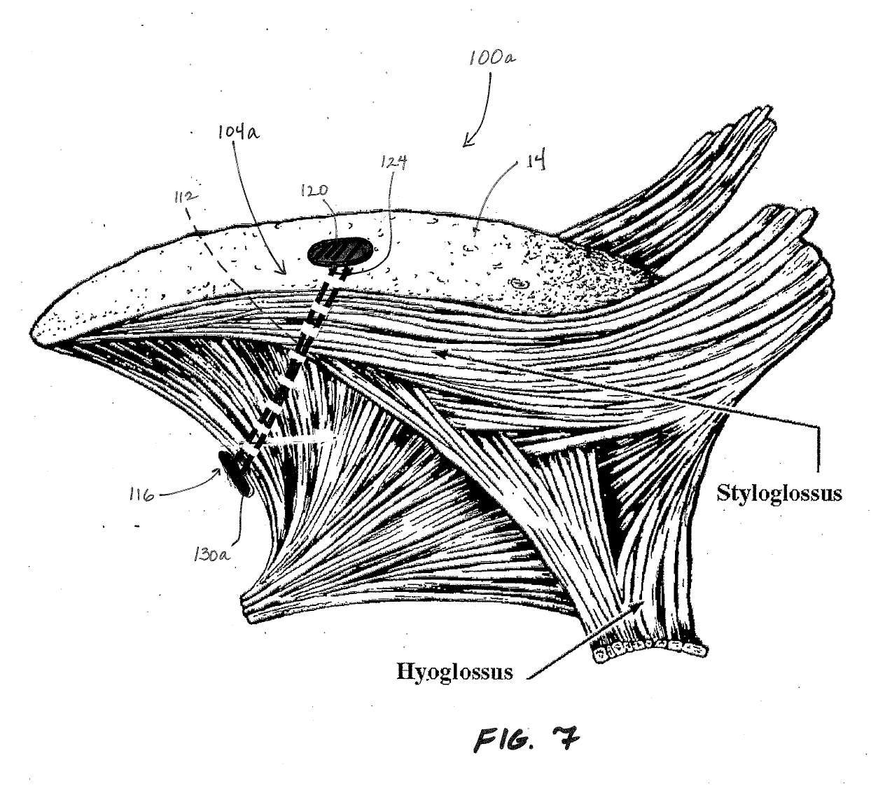

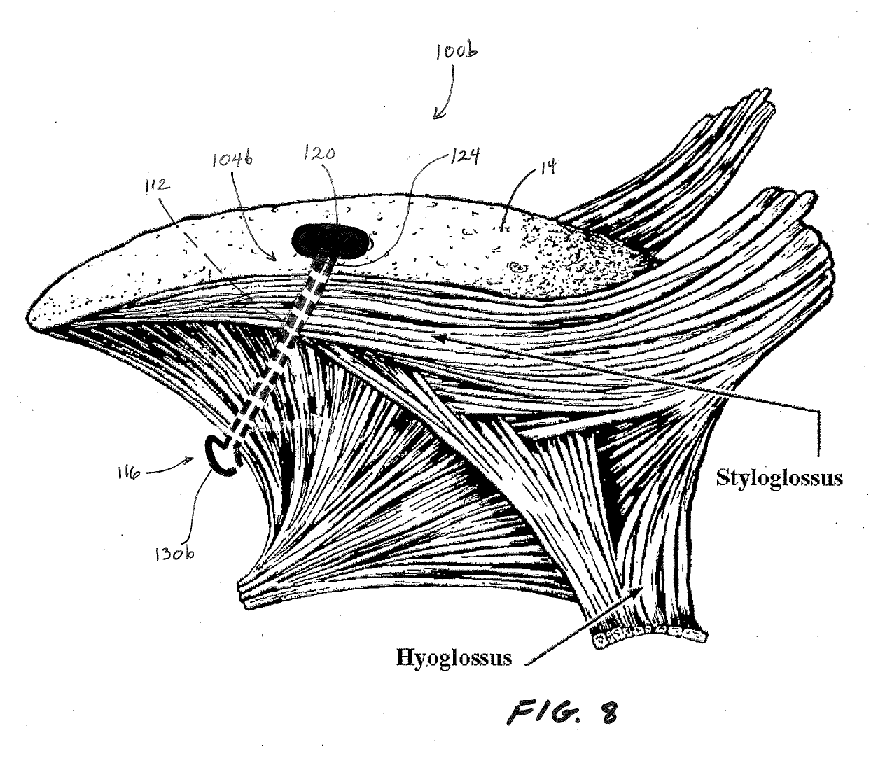

Referring now to FIG. 7, shown therein and designated by the reference numeral 100a is a portion of a tongue-stabilization device. Device 100a comprises a body 104a configured to be coupled to a retention member (described below). Body 104a has an elongated portion 112 with a coupling end 116 and an enlarged end 120, and is configured to be inserted into a hole 124 through a tongue 14 of a human patient (e.g., 18). Enlarged end 120 is configured to resist being pulled through hole 124 (e.g., enlarged end 120 is larger than hole 124). In the embodiment shown, enlarged end 120 has a rounded, flattened shape that is larger than hole 124. Enlarged end 120 can also be configured to resist chafing, scraping, and/or cutting the tongue and/or other portions of a patient's mouth. More specifically, enlarged portion 120 (and/or other portions, members, or parts of device 100a) can be formed without sharp corners, with smooth surfaces, and/or of flexible or resilient materials to reduce the likelihood of any damaging interaction with the patients tongue or mouth. In the embodiment shown, coupling end 116 includes a connection member 130 to permit body 104a to be coupled to retention member (described below). In the embodiment shown, connection member 130a comprises a loop. In other embodiments, connection member 130a can comprise any suitable structure or shape configured to be coupled (e.g., removably) to any of the present retention members (described below). In the embodiment shown, loop 130a is larger than hole 124, but does not necessarily need to be larger than hole 124. Body 104a can be placed (e.g., hole 124 can be formed in tongue and/or body 104a inserted in hole 124) under local anesthesia, such that, for example, any of the present retention members 108c-108o (shown in FIGS. 9-21, discussed below) can be coupled to body 104a (e.g., at night) to anchor tongue 14 to the mandibular teeth to prevent the tongue from collapsing or falling posteriorly and/or impeding the airway (e.g., during sleep).

FIG. 8 depicts an alternate embodiment 104b of body 104a. Body 104b is substantially similar to body 104a, with the primary exception that body 104b comprises a clasp 130b that is configured to be coupled (e.g., removably) to a corresponding structure (e.g., a loop or ring) of a retention member (described below). For example, clasp 130b can be configured to be opened and/or closed by a user (e.g., a patient).

Referring now to FIGS. 9-21, various views are shown of embodiments of the present tongue-stabilization devices. The various embodiments shown in FIGS. 9-21 are similar to one another in various respects, and the differences are therefore primarily described.

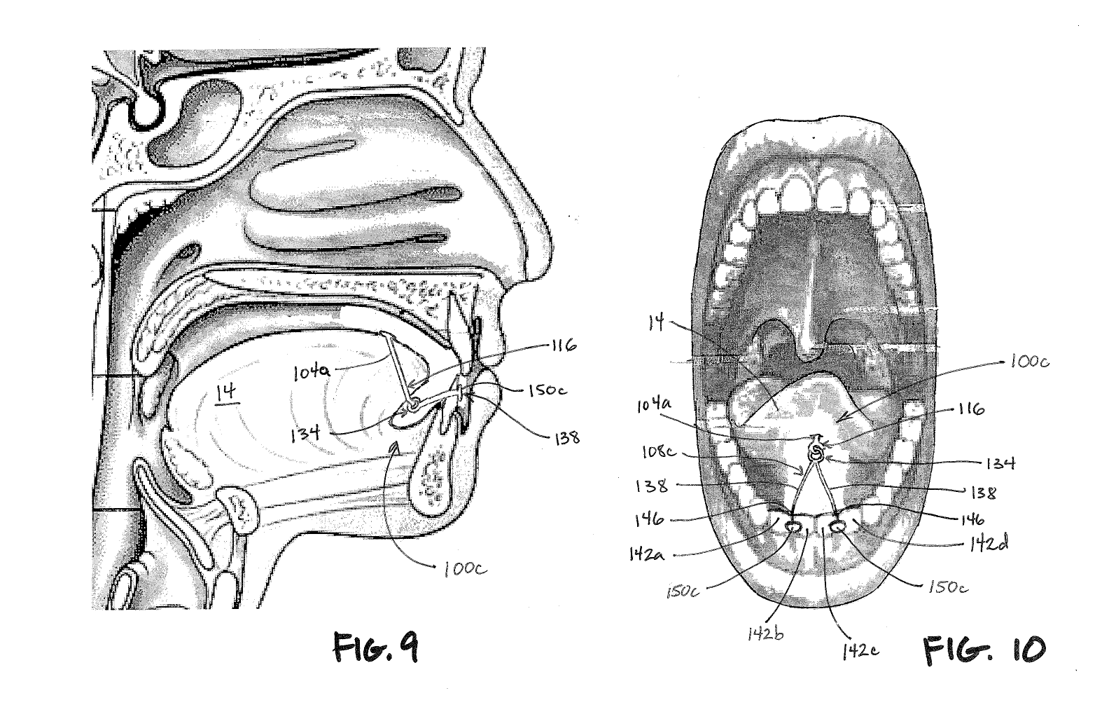

FIG. 9 depicts a side cross-sectional view of one of the present tongue-stabilization devices 100c, and FIG. 10 depicts a front view of device 100c. Device 100c comprises a body 104a (having an elongated portion with a coupling end and an enlarged end, the body configured to be inserted into a hole through the tongue of a human patient, and the enlarged end configured to resist being pulled through the hole, as described with reference to FIG. 7). Device 100c further comprises a retention member 108c having a coupling portion 134 and one or more (e.g., two) tooth portions 138. Coupling portion 134 is configured to be coupled to coupling end 116 of body 104a, and each tooth portion 138 is configured to fit between a set of two laterally-adjacent teeth of the patient (e.g., 142a and 142b, or 142c and 142d). Each tooth portion 138 comprises a narrow portion 146 configured to fit between two laterally-adjacent teeth (e.g., 142a and 142b, or 142c and 142d), and an enlarged portion 150c configured to not fit between the two laterally-adjacent teeth (e.g., configured to be larger than the space between two laterally adjacent teeth and/or otherwise shaped such that enlarged portion 150c will not fit between the laterally-adjacent teeth). In the embodiment shown, retention member 108c comprises two tooth portions 138, each configured to fit between a different set of two laterally-adjacent teeth of the patient (e.g., 142a and 142b, or 142c and 142d). In this way, each tooth portion 138 is configured such that a user can slide narrow portion 146 between two laterally-adjacent teeth such that enlarged portion 150c is near or in contact with the respective laterally-adjacent teeth (e.g., 142a and 142b). Tooth portions 138 can be unitary (e.g., formed of a single piece) with one another and/or with coupling portion 134. In this way, when device 100a is installed as shown, body 104a and retention member 108c cooperate to hold tongue 14 in a forward position (e.g., to prevent the tongue from moving rearward beyond a predetermined position, such as, for example, as may be predetermined by the position of hole 124 in tongue 14 and the size of device 100a).

Coupling portion 134 can comprise one or more of a ring, clasp, or the like configured to be coupled (e.g., removably) to coupling end 116 of body 104a. In the embodiment shown, coupling end 116 of body 104a comprises an opening (e.g., has an opening defined therethrough, such as, for example, in a ring, or a hole formed through coupling end 116 of body 108c), and coupling portion 134 of retention member 108c comprises a ring configured to be coupled (e.g., removably) to the opening (in the coupling end of the body).

Body 104a and/or retention member 108c can comprise any suitable biocompatible and/or hypoallergenic material or materials, such as, for example, stainless steel, titanium, polymer, composite, or the like. Body 104a and retention member 108c need not comprise the same material or materials. For example, body 104a can comprise a substantially rigid material such as stainless steel, and/or retention member 108c can comprise a resilient material such as a resilient polymer (or retention member 108c can comprise a substantially rigid material such as stainless steel, and body 104a can comprise a resilient material such as a resilient polymer). In this way, stress and/or wear on teeth can be limited and/or reduced.

It is understood that the embodiments shown in FIGS. 9-11 are merely exemplary embodiments of the present disclosure. Other embodiments for example, may comprise variations or alterations to the components illustrated. For example, in certain embodiments, the retention member may be configured to attach or couple to the teeth or on the teeth. In specific embodiments, the retention member may be a custom-fitted, removable dental appliance that fits over the teeth, a permanent appliance that is cemented or otherwise affixed directly to the teeth or any device that rests between the teeth or slips around one tooth or multiple teeth.

FIG. 11 depicts another embodiment 100d of the present tongue-stabilization devices. Device 100d comprises a body 104a and a retention member 108d having a coupling portion 134 and a single tooth portion 138 (configured to fit between a single set of two laterally-adjacent teeth 142b and 142c). As shown, enlarged portion 150d of tooth portion 138 of device 100d comprises a ring that is configured to not fit between laterally-adjacent teeth 142a and 142b.

FIG. 12 depicts another embodiment 100e of the present tongue-stabilization devices. Device 100e comprises a body 104e that is similar to body 104a with the primary exception that body 104e comprises a ring 130e that is substantially larger than elongated portion 112. In the embodiment shown, body 104e can comprise a flexible and/or resilient material, such that ring 130e can be compressed to fit through hole 124 in tongue 14; and such that once through hole 124, ring 130e will relax or expand to an enlarged and relaxed state in which ring 130e is larger than hole 124 to resist ring 130e being pulled through hole 124 (e.g., resists to a lesser degree than enlarged end 120 of body 104e resists being pulled through hole 124. Additionally, enlarged portion 150e of tooth portion 138 of body 104e comprises a flattened tab that extends laterally a distance (e.g., greater than half the width of an adjacent tooth 142b or 142c) in each of left and right directions.

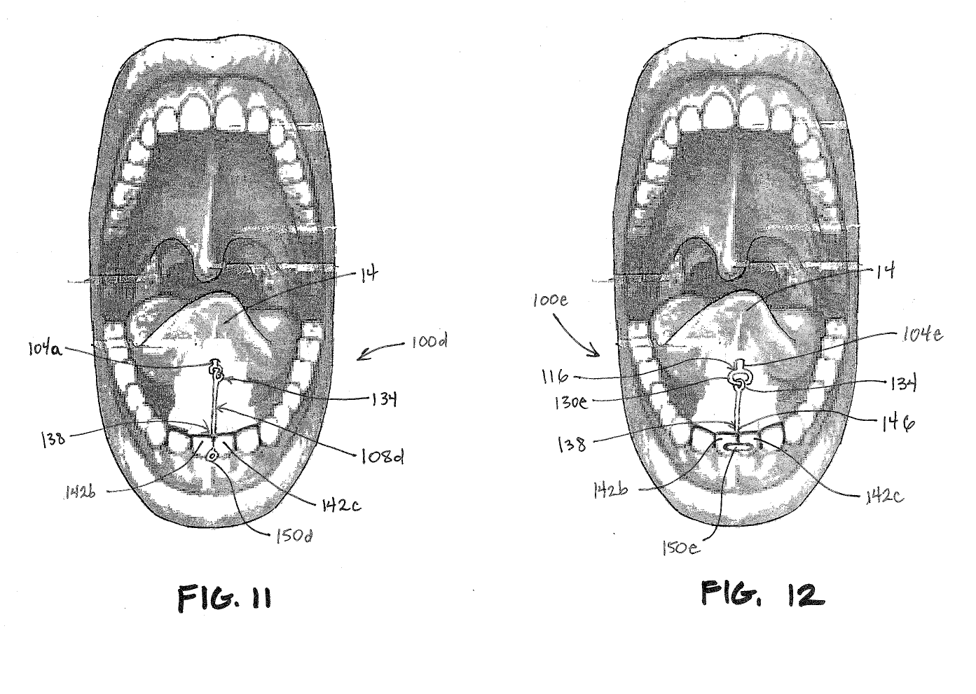

FIG. 13 depicts another embodiment 100f of the present tongue-stabilization devices. Device 100f comprises a body 104f that is similar to body 104a with the primary exception that body 104f comprises a connection structure 130f that does not comprise a ring. In the embodiment shown, connection structure 130f has a diamond shape. In some embodiments connection structure 130f comprises an axial (axially aligned with elongated portion 112) opening (e.g., threaded, or knurled) into which coupling portion 134 of retention member 108f can be inserted. For example, in such embodiments, coupling portion 134 can be elongated and/or can be configured (e.g., can be threaded or knurled) to correspond to the threads or knurls of an axial opening. In other embodiments, connection structure 130f (and therefore body 104f) can be unitary with retention member 108f, such that, for example, the enlarged connection structure 130f merely comprises an enlarged portion configured to resist motion of body 104f relative to tongue 14 when body 104f is disposed in tongue 14, as shown. Stated another way, connection structure 130f is configured to resist movement of tongue 14 relative to body 104f. In some embodiments, connection structure 130f is configured to at least partially collapse when pressed through hole 124, and to relax or de-compress (e.g., enlarge) when connection structure 130 is pushed out of hole 124. In other embodiments, connection structure 130f is substantially rigid such that as connection structure 130f is pushed through hole 124, the soft tissue of the tongue is pushed apart such that hole 124 temporarily expands, and then hole 124 contracts once coupling portion 130f is pushed out of hole 124. Enlarged portion 150f is substantially similar to enlarged portion 150e of device 100e.

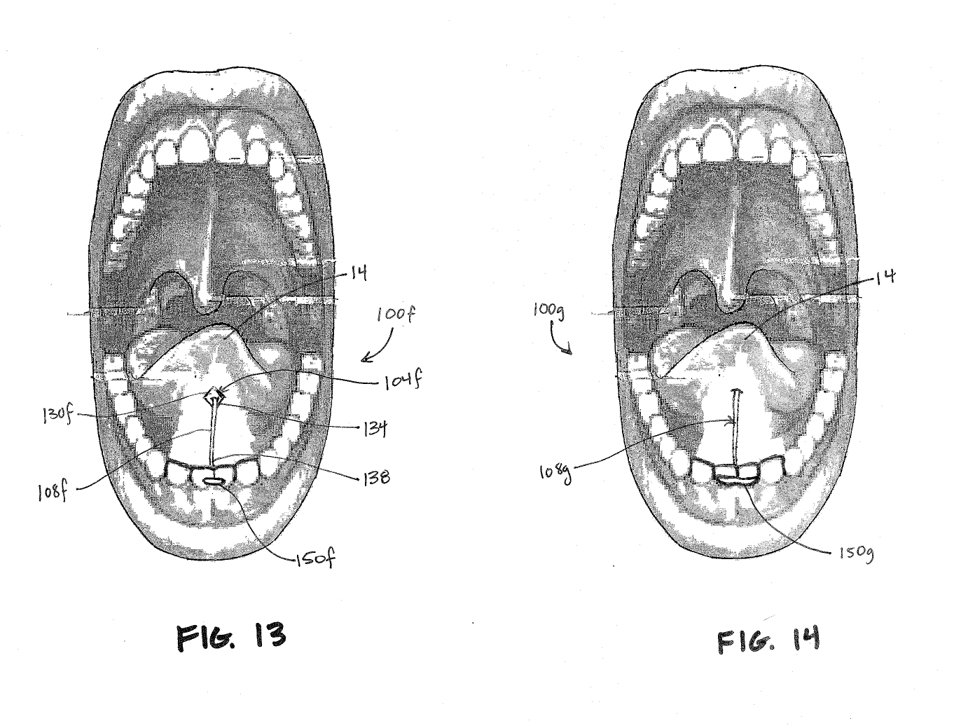

FIG. 14 depicts another embodiment 100g of the present tongue-stabilization devices. Device 100g comprises a body 104g that is similar to body 104a with the primary exception that body 104g does not have a connection structure, and is instead unitary with retention member 108g. In particular, there is no enlarged or other structure designating or demarcating a border or distinction between body 104g and retention member 108g. Retention member 108g comprises an enlarged portion 150g of tooth portion 138 that comprises a flattened tab that extends laterally a distance (e.g., about the width of an adjacent tooth 142b or 142c) in each of left and right directions. Enlarged portion 150 can be shaped to contour to an outer surface of each tooth 142b, 142c.

FIG. 15 depicts another embodiment 100h of the present tongue-stabilization devices. Device 100h comprises a body 104h that is similar to body 104f, and a retention member 108h that is substantially similar to retention member 108f, with the primary exceptions that body 104h comprises a connection structure 130h that has a square shape, and retention member 108h has an enlarged end 150h that comprise a ring (e.g., substantially similar to that of retention member 108d).

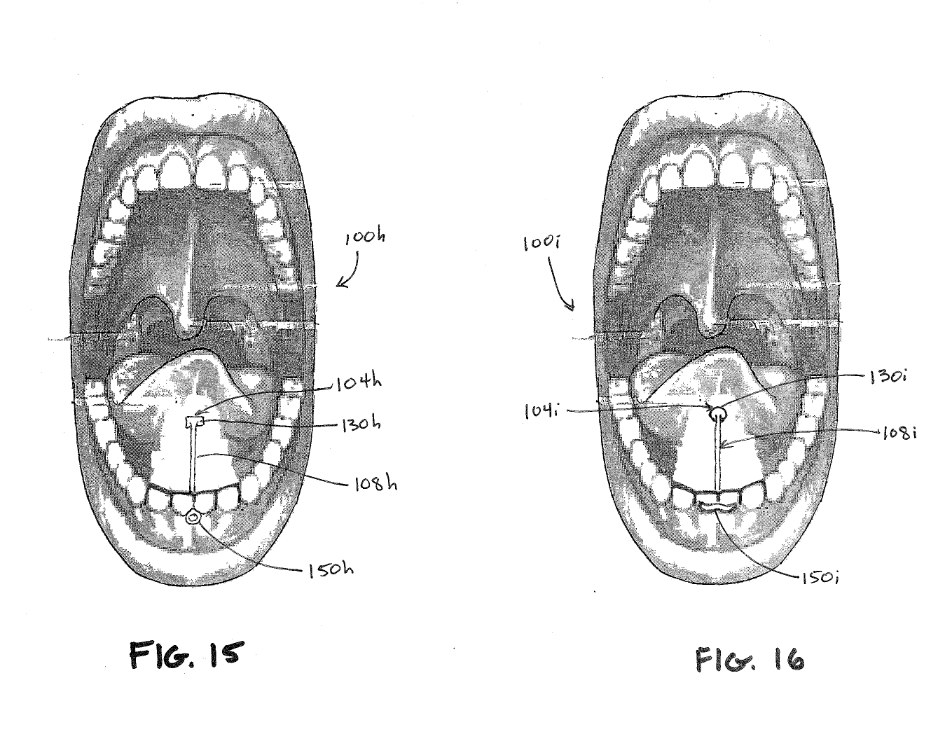

FIG. 16 depicts another embodiment 100i of the present tongue-stabilization devices. Device 100i comprises a body 104i that is similar to body 104f, and a retention member 108i that is substantially similar to retention member 108f, with the primary exceptions that body 104i comprises a connection structure 130i that has a round or circular shape.

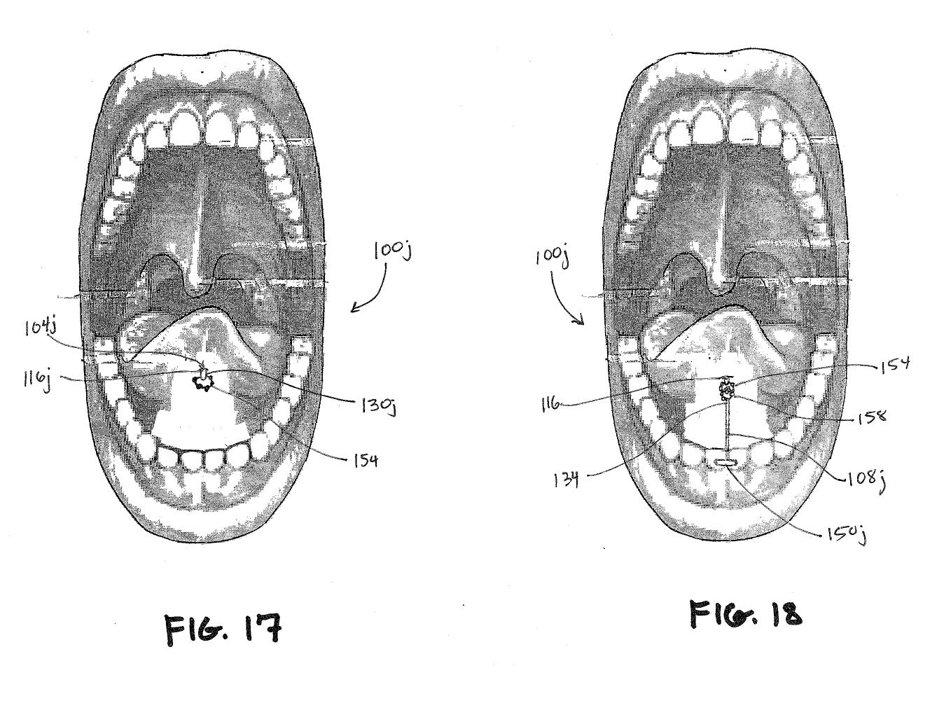

FIGS. 17 and 18 depict a another embodiment 100j of the present tongue-stabilization devices. Device 100j comprises a body 104j that is similar to body 104a, with the primary exceptions that the connection end 116 of body 104j has a maximum transverse dimension (e.g., diameter or width perpendicular to the length of elongated portion 112) that is substantially the same as or less than the maximum transverse dimension (e.g., diameter or width) as elongated portion 112 of the body. Connection structure 130j comprises an opening extending through connection end 116 of body 104j, and a coupling member 154 configured to be coupled to coupling end 116 (e.g., through opening 130j) of body 104j and coupling portion 134 of retention member 108j. For example, in the embodiment shown, coupling portion 134 of retention member 108j comprises an opening through which coupling member 154 can be passed to couple body 104j to retention member 108j, or through which a second coupling member 158 can be passed such that second coupling member 158 can be coupled to coupling member 154. In some embodiments, coupling member 154 and/or coupling member 158 can comprise a small chain (e.g., comprising stainless steel, titanium, or the like). Enlarged portion 150j is substantially similar to enlarged portion 150e of device 100e.

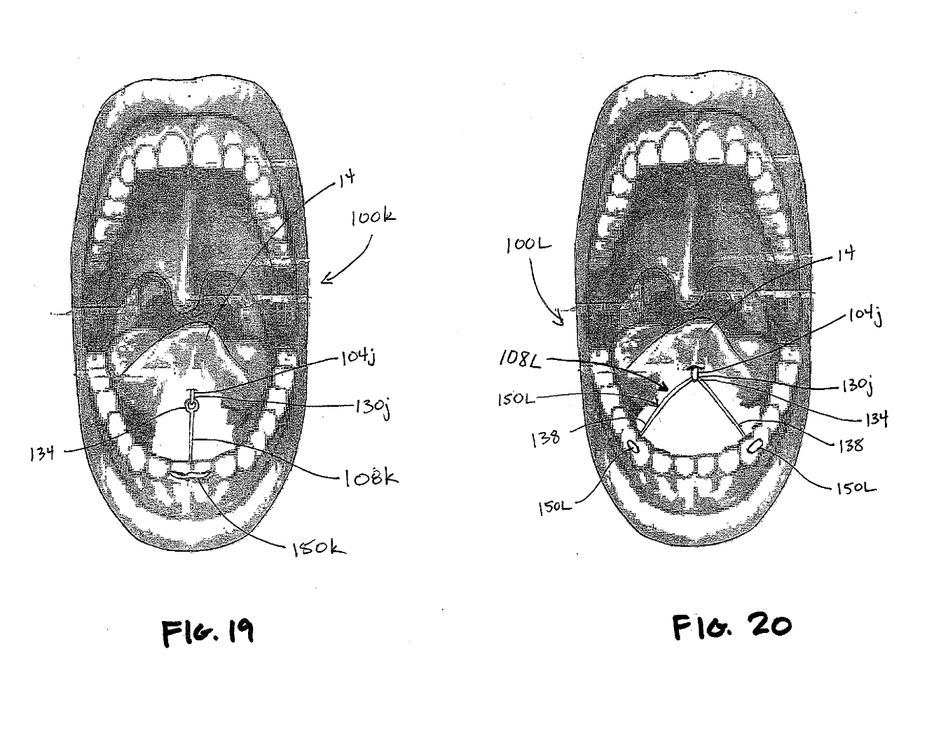

FIG. 19 depicts another embodiment 100k of the present tongue-stabilization devices. Device 100k comprises a body 104j. Device 100k further comprises a retention member 108k that is substantially similar to retention member 108e, with the primary exception that enlarged portion 150k is substantially similar to enlarged portion 150g of device 100g. In some embodiments, coupling portion 134 of retention member 108k can comprise a clasp or the like that is configured to be removably coupled to opening 130j. In other embodiments coupling portion 134 (e.g., ring) of retention member 108k is permanently or non-removably (without destroying retention member 108) coupled to body 104j.

FIG. 20 depicts another embodiment 100L of the present tongue-stabilization devices. Device 100L comprises a body 104j. Device 100L further comprises a retention member 108L that is substantially similar to retention member 108c, with the primary exception that retention member 108L comprises a coupling portion 134 (e.g., rather than a ring) that is the midpoint between the enlarged portions 150L of the two tooth portions 138. In the embodiment shown, the two tooth portions 138 of retention member 108L are unitary.

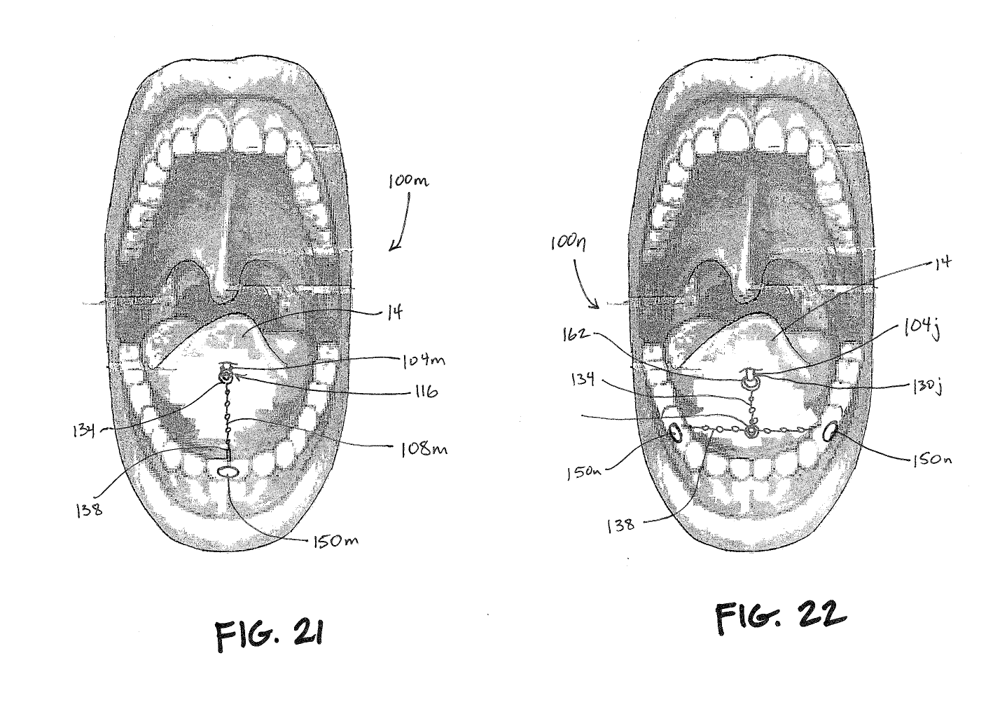

In some embodiments of the present tongue-stabilization devices, elongated portion 112 an/or connection end 116 of the body comprise chain. In some embodiments, the retention member comprises chain. For example, FIG. 21 depicts another embodiment 100m of the present tongue-stabilization devices. Device 100m comprises a body 104m and a retention member 108m that are substantially similar to body 104e and retention member 108e, with the primary exceptions that elongated portion 112 and connection end 116 of body 104m comprise a chain looped from (and back to) enlarged end 120, and retention member 108m comprises a chain extending between tooth portion 138 and coupling portion 134. Coupling portion 134 can comprise a ring through which the chain of connection end 116 can extend through, or coupling portion 134 can comprise a clasp or the like for coupling (e.g., removably) to connection 116 end of body 104m. In some embodiments, coupling portion 134 can comprise a ring through which the chain of connection end 116 can extend through, or coupling portion 134 can comprise a clasp or the like for coupling (e.g., removably) to connection 116 of body 104m.

By way of another example, FIG. 22 depicts another embodiment 100n of the present tongue-stabilization devices. Device 100n comprises a body 104j. Device 100n further comprises a retention member 108n that is similar to retention member 108L, with the primary exceptions that coupling portion 134 comprises a length of chain configured to be coupled to body 104j (e.g., by a ring 162 through opening 130j) and to be coupled to tooth portion 138 (e.g., by a ring 166), and that tooth portion 138 comprises a piece of chain having an enlarged portion 150n at either end. In some embodiments coupling portion 134 is coupled directly (e.g., non-removably to tooth portion 138). Stated another way, and as shown in FIG. 22, retention member 108n is configured such that if worn by a patient at least a portion of retention member 108n (e.g., tooth portion 138) extends linearly across the lower jaw of the patient substantially perpendicular to elongated portion 112 of body 104j).

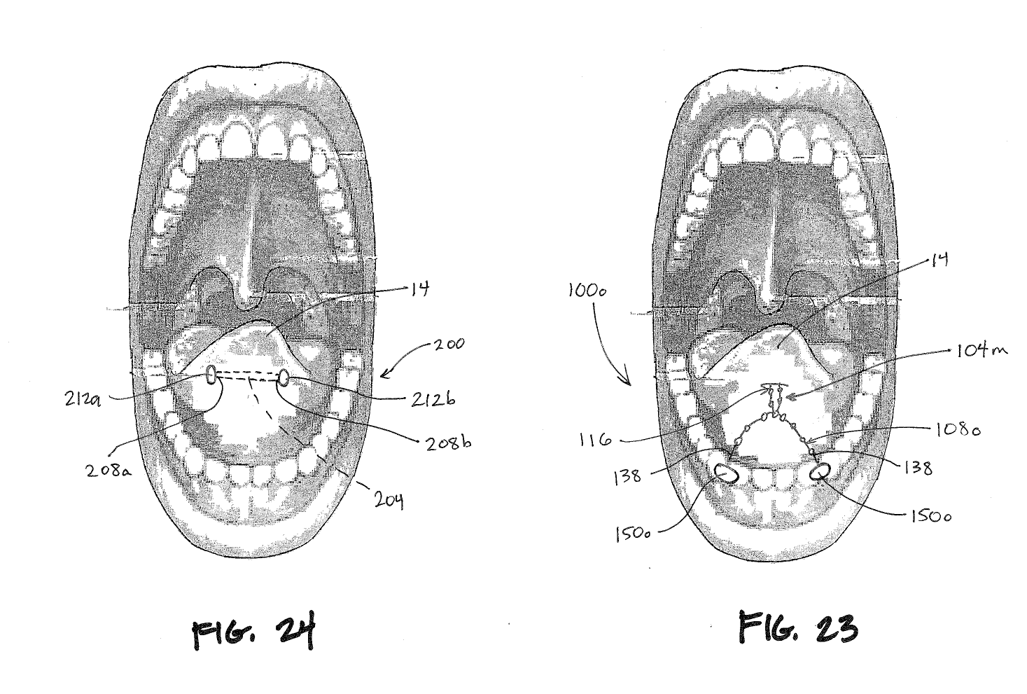

By way of another example, FIG. 23 depicts another embodiment 100o of the present tongue-stabilization devices. Device 100o comprises a body 104m. Device 100o further comprises a retention member 108o that is substantially similar to retention member 108L, with the primary exception that retention member 108o comprises a length of chain extending between tooth portions 138 (e.g., enlarged portions 150o).

Some of the present methods of stabilizing a tongue comprise: providing a tongue-stabilization device (e.g., 100a-100o) comprising a body and a retention member; positioning the body through a hole (e.g., 124) in the tongue (e.g., 14) of a patient (e.g., 18); coupling a coupling portion of the retention member to a coupling end of the body; and positioning each of the one or more tooth portions of the retention member between a set of two laterally-adjacent teeth (e.g., 142a and 142b, or 142c and 142d) of the patient (e.g., 18) such that the device 100a resists posterior motion of the tongue.

Referring now to FIG. 24, an embodiment 200 is shown of one of the present tongue-shaping devices. Device 200 comprises: a bar 204 configured to extend transversely across tongue 14 of a patient through two holes 208a, 208b in the tongue. In the embodiment shown, device 200 further comprises enlarged ends 212a and 212b that are larger than (have a diameter or other maximum transverse dimension that is larger than) the diameter or other corresponding dimension of holes 208a, 208b such that if device 200 is installed in a tongue as shown in FIG. 23, enlarged ends 212a, 212b will resist being pulled through holes 208a, 208b. In the embodiment shown, holes 208a and 208b extend through the tongue from (and through) a lower surface of the tongue to (and through) an upper surface of the tongue. As shown, in some methods of shaping a tongue with device 200, device 200 is disposed through the tongue such that bar 204 extends across an upper surface of the tongue and such that enlarged ends 212a, 212b are disposed below the lower surface of the tongue. In other embodiments, device 200 can be disposed relative to the tongue such that enlarged ends 212a, 212b are disposed on or above an upper surface of the tongue and bar 204 extends below the lower surface of the tongue. In some embodiments, enlarged ends 212a, 212b each have a maximum transverse dimension (e.g., diameter) that is larger than the corresponding maximum transverse dimension (e.g., diameter) of each of holes 208a, 208b such that enlarged ends 212a and 212b resist motion of bar 204 relative to the tongue. In this way, device 200 can be inserted through holes 208a, 208b to compress tongue 14, such as, for example, to compress and/or shape the tongue to prevent the tongue from blocking an airway of a patient and/or reduce blockage of the airway by the tongue. One or both of enlarged ends 212a, 212b can be configured to thread or screw into and/or out of bar 204 such that an enlarged end (e.g., 212a) can be removed from bar 204, bar 204 inserted through holes 208a and 208b, and the enlarged end (e.g., 212a) re-attached or re-coupled to bar 204 to prevent bar 204 from pulling out of holes 208a, 208b. Device 200 can comprise any suitable materials and/or be configured to have any suitable characteristics, such as, for example, any of the materials and/or characteristics described above for devices 10 and/or 100a-100o.

The various illustrative embodiments of devices, systems, and methods described herein are not intended to be limited to the particular forms disclosed. Rather, they include all modifications and alternatives falling within the scope of the claims.

The claims are not intended to include, and should not be interpreted to include, means-plus- or step-plus-function limitations, unless such a limitation is explicitly recited in a given claim using the phrase(s) “means for” or “step for,” respectively.

### REFERENCES

The following references, to the extent that they provide exemplary procedural or other details supplementary to those set forth herein, are specifically incorporated herein by reference.
U.S. Pat. No. 5,245,023 “Method for producing novel polyester biopolymers” by Peoples and Sinskey, issued Sep. 14, 1993.
U.S. Pat. No. 5,250,430 “Polyhydroxyalkanoate polymerase” by Peoples and Sinskey, issued Oct. 5, 1993.
U.S. Pat. No. 5,534,432 “Polyhydroxybutyrate polymerase” by Peoples and Sinskey, issued Jul. 9, 1996.
U.S. Pat. No. 5,663,063 “Method for producing polyester biopolymers” by Peoples and Sinskey, issued Sep. 2, 1997.
U.S. Pat. No. 6,245,537 “Removing endotoxin with an oxidizing agent from polyhydroxyalkanoates produced by fermentation” by Williams, Martin, Horowitz and Gerngross, issued Jun. 12, 2001.
U.S. Pat. No. 6,316,262 “Biological systems for manufacture of polyhydroxyalkanoate polymers containing 4-hydroxyacids” by Huisman, Skraly, Martin and Peoples, issued Nov. 13, 2001.
U.S. Pat. No. 6,514,515 “Bioabsorbable, biocompatible polymers for tissue engineering” by Williams, issued Feb. 4, 2003.
U.S. Pat. No. 6,548,569 “Medical devices and applications of polyhydroxyalkanoate polymers” by Williams, Martin, and Skraly, issued Apr. 15, 2003. U.S. Pat. No. 6,555,123 “Polyhydroxyalkanoate compositions for soft tissue repair, augmentation, and viscosupplementation, by Williams and Martin, issued Apr. 29, 2003.
U.S. Pat. No. 6,585,994 “Polyhydroxyalkanoate compositions for soft tissue repair, augmentation, and viscosupplementation, by Williams and Martin, issued Jul. 1, 2003.
U.S. Pat. No. 6,592,892 “Flushable disposable polymeric products” by Williams, issued Jul. 15, 2003.
U.S. Pat. No. 6,593,116 “Transgenic microbial polyhydroxyalkanoate producers” by Huisman, Peoples, and Skraly, issued Jul. 15, 2003.
U.S. Pat. No. 6,610,764 “Polyhydroxyalkanoate compositions having controlled degradation rates” by Martin, Skraly, and Williams, issued Aug. 26, 2003.
U.S. Pat. No. 6,623,749 “Medical device containing polyhydroxyalkanoate treated with oxidizing agent to remove endotoxin” by Williams, Martin, Gerngross and Horowitz, issued Sep. 23, 2003.
U.S. Pat. No. 6,689,589 “Biological systems for manufacturing of polyhydroxyalkanoates polymers containing 4-hydroxyacids” by Huisman, Skraly, Martin and Peoples, issued Feb. 10, 2004.
U.S. Pat. No. 6,746,685 “Bioabsorbable, biocompatible polymers for tissue engineering” by Williams, issued Jun. 8, 2004.
U.S. Pat. No. 6,828,357 “Polyhydroxyalkanaote compositions having controlled degradation rates” by Martin, Skraly, and Williams, issued Dec. 7, 2004.
U.S. Pat. No. 6,838,493 “Medical devices and applications of polyhydroxyalkanoate polymers” by Williams, Martin, and Skraly, issued Jan. 4, 2005.
U.S. Pat. No. 6,867,247 “Medical devices and applications of polyhydroxyalkanoate polymers” by Williams, Martin, Skraly, issued Mar. 15, 2005.
U.S. Pat. No. 6,867,248 “Polyhydroxyalkanoate compositions having controlled degradation rates” by Martin, Skraly, and Williams, issued Mar. 15, 2005.
U.S. Pat. No. 6,878,758 “Polyhydroxyalkanoate compositions having controlled degradation rates” by Martin, Skraly and Williams, issued Apr. 12, 2005.
US Pub App. No. 20020164729 “Production of polyhydroxyalkanoates from polyols” by Skraly and Sholl, published Nov. 7, 2002.
US Pub. App. No. 20040234576 “Polyhydroxyalkanoate medical textiles and fibers” by Martin, Rizk, Ahuja, and Williams, published Nov. 25, 2004.

1. A tongue stabilization device, comprising:
an anchor having an anterior penetration point, a posterior end, and a plurality of barbs each having a point extending away from the penetration point;
a suture connected to the anchor; and
a counter-tension member configured to be slidably disposed on the suture.

2. The tongue-stabilization device of claim 1, where the counter-tension member is configured to freely slide relative to the suture in a first direction, and to resist sliding relative to the suture in a second direction.

3. The tongue-stabilization device, where at least one of the anchor, suture, and counter-tension member comprises a bio-absorbable material.

4. The tongue-stabilization device of claim 3, where each of the anchor, suture, and counter-tension member each comprise a bio-absorbable material.

5. A method of stabilizing a tongue of a patient, the method comprising:
providing a tongue-stabilization device comprising:
an anchor configured such that if the anchor is inserted into soft tissue of a patient the anchor will resist removal from the soft tissue;
a suture having a first end connected to the anchor, and a second end extending from the anchor; and
a counter-tension member configured to be slidably disposed on the suture;
creating an incision through the skin in a submental area of the patient;
inserting the tongue-stabilization device through the incision such that the anchor is disposed near a posterior portion of the tongue and the suture extends through the anterior portion of the tongue and through the incision;
positioning the counter-tension member along the suture outside the tongue adjacent a lower surface of the tongue; and
tensioning the suture between the anchor and the counter-tension member.

6. The method of claim 5, where inserting comprises:
disposing at least a portion of the tongue-stabilization device in a needle;
inserting the needle through the incision such that the anchor is disposed near a posterior portion of the tongue and the needle extends through the anterior portion of the tongue and through the incision; and
removing the needle from the tongue.

7. The method of claim 6 further comprising:
creating a void in the tongue proximal to the anchor, wherein the void is oriented generally parallel to an axis extending from the posterior portion of the tongue to the anterior portion of the tongue prior to tensioning the suture; and
the void is oriented generally perpendicular to an axis extending from the posterior portion of the tongue to the anterior portion of the tongue after tensioning the suture.

8. The method of claim 5, where inserting comprises:
disposing at least a portion of the tongue-stabilization device in an insertion device, wherein the insertion device comprises an ablation mechanism;
inserting the insertion device through the incision such that the anchor is disposed near a posterior portion of the tongue and the insertion device extends through the anterior portion of the tongue and through the incision;
ablating tissue with the ablation mechanism; and
removing the insertion device from the tongue.

9. The method of claim 5, where the step of tensioning the suture is performed before the step of positioning the counter tension member.

10. The method of claim 5, where the step of positioning the counter-tension member comprises sliding the counter-tension member along the suture.

11. The method of claim 10, where the counter-tension member is configured such that if the counter-tension member is disposed on the suture the counter-tension member will freely slide relative to the suture in a first direction, and resist sliding relative to the suture in a second direction.

12. The method of claim 5, where the anchor has an anterior penetration point, a posterior end, a longitudinal axis extending from the, and a plurality of barbs each having a point extending away from the penetration point.

13. The method of claim 5, where the suture comprises a plurality of barbs extending away from the anchor.

14. The method of claim 5, where the anchor, suture, and counter-tension member each comprise a bio-absorbable material.

15. A tongue-stabilization device, comprising:
a body having an elongated portion with a coupling end and an enlarged end, the body configured to be inserted into a hole through the tongue of a human patient, and the enlarged end configured to resist being pulled through the hole; and
a retention member having a coupling portion and a tooth portion, the coupling portion configured to be coupled to the coupling end of the body, and the tooth portion configured to couple to one or more teeth of the patient.

16. The tongue-stabilization device of claim 15, where the coupling end of the body comprises an opening, the coupling portion of the retention member comprises a ring configured to be coupled to the opening.

17. The tongue-stabilization device of claim 15, where the ring is configured to be removably coupled to the opening.

18. The tongue-stabilization device of claim 15, where the retention member comprises a narrow portion configured to fit between two laterally-adjacent teeth, and an enlarged portion configured to not fit between the two laterally-adjacent teeth.

19. The tongue-stabilization device of claim 15, where the retention member comprises two tooth portions each configured to fit between a different set of two laterally-adjacent teeth of the patient.

20. The tongue-stabilization device of claim 19, where each tooth portion comprises a narrow portion configured to fit between two laterally-adjacent teeth, and an enlarged portion configured to not fit between the two laterally-adjacent teeth.

21. The tongue-stabilization device of claim 19, where the retention member is configured is configured such that if worn by a patient at least a portion of the retention member extends linearly across the lower jaw of the patient substantially perpendicular to the elongated portion of the body.

22. The tongue-stabilization device of claim 21, where the two tooth portions of the retention member are unitary, and the coupling portion is the midpoint between the enlarged portions of the two tooth portions.

23. The tongue-stabilization device of claim 15, where the body and the retention member are unitary.

24. The tongue stabilization device of claim 15, where the coupling portion of the body comprises an enlarged portion configured to resist movement of the tongue of a patient relative to the body.

25. The tongue-stabilization device of claim 15, where the coupling end of the body has a maximum transverse dimension that is substantially the same as or less than the maximum transverse dimension as the elongated portion of the body.

26. The tongue-stabilization device of claim 25, further comprising:
a coupling member configured to be coupled to the coupling end of the body and the coupling portion of the retention member.

27. The tongue-stabilization device of claim 26, where the coupling member comprises chain.

28. The tongue-stabilization device of claim 15, where the elongated portion and coupling end of the body are each flexible.

29. The tongue-stabilization device of claim 28, where the elongated portion and coupling end of the body are unitary.

30. The tongue-stabilization device of claim 29, where the elongated portion and coupling end of the body comprise chain.

31. The tongue-stabilization device of claim 15, where the retention member comprises chain.

32. A method of stabilizing a tongue of a patient, the method comprising:
providing a tongue-stabilization device comprising:
a body having an elongated portion with a coupling end and an enlarged end, the body configured to be inserted into a hole through the tongue of a human patient, and the enlarged end configured to resist being pulled through the hole; and
a retention member having a coupling portion and a tooth portion, the coupling portion configured to be coupled to the coupling end of the body, and the tooth portion configured to couple to one or more teeth of the patient;
positioning the body of the tongue-stabilization device through a hole in the tongue of a patient;
coupling the coupling portion of the retention member to the coupling end of the body of the tongue-stabilization device; and
positioning each of the one or more tooth portions of the retention member between a set of two laterally-adjacent teeth of the patient such that the tongue-stabilization device resists posterior motion of the tongue.
